(19) United States
(12) Patent Application Publication
(10) Pub. No.: US 2014/0199255 A1
(43) Pub. Date: Jul. 17, 2014

(54) Use Of Natural Or Naturally Derived Polyesters As Hair Fixatives

(71) Applicants: Camille T. Sasik, Minnetonka, MN (US); Cindy L. Orr, Blaine, MN (US)

(72) Inventors: Camille T. Sasik, Minnetonka, MN (US); Cindy L. Orr, Blaine, MN (US)

(21) Appl. No.: 13/924,409

(22) Filed: Jun. 21, 2013

Related U.S. Application Data
(60) Provisional application No. 61/663,214, filed on Jun. 22, 2012.

(51) Int. Cl. A61K 8/85; A61Q 5/06

(52) U.S. Cl. CPC A61K 8/85; A61Q 5/06
USPC 424/70.9; 424/70.11

(57) ABSTRACT

The invention relates to a use of a composition comprising at least one naturally derived poly(lactic acid) film-forming polymer and a carrier for styling mammalian hair.

### CROSS-REFERENCE TO RELATED APPLICATIONS

The present application claims priority from U.S. Provisional Application No. 61/663,214, filed Jun. 22, 2012.

### FIELD OF THE INVENTION

The invention relates to the use of a composition comprising at least one naturally derived poly(lactic acid) film-forming polymer and a carrier for styling mammalian hair. Specifically, the present invention relates to the use of naturally derived poly(lactic acid) which may be used as hair fixative agents.

### BACKGROUND OF THE INVENTION

Cosmetic compositions incorporate a variety of polymers to provide aesthetic and feel benefits to consumers. Common polymers providing such benefits include poly(vinylpyrrolidone), polyquaternium-10, polyquaternium-11, and polyquaternium-16. In recent years, development of new synthetic polymers has enhanced overall product performance and benefit efficacy. And additional benefits from silicone elastomer technology have created new avenues for delivering improved benefits to consumers.

In view of the rapid worldwide depletion of petrochemical feedstocks, attention has increasingly turned to the production of new, useful and environmentally friendly polymers which would offer a more sustainable future. But despite developments in the synthetic polymer space, research in the natural polymer space has been comparatively limited.

Starch derivatives have been developed which have good film forming properties and show promising hold benefits for styling formulations. But they are generally expensive and thereby limit commercial formulation possibilities.

Chitosan is abundantly available in nature. But it tends to be limited with regard to its solubility properties. In order to improve solubility, attempts have been made to create mixtures including chitosan and PVP/VA. Other techniques involve dissolving chitosan in fatty acids or fatty alcohols to develop cosmetic formulations. In general, it appears difficult to formulate chitosan in systems without synthetic ingredients.

Poly(lactic acids) (PLA) are well known for fibrous, packaging, and biomedical applications. PLA is a biodegradable linear aliphatic thermoplastic polyester derived from 100% renewable sources, such as corn and sugar beet. Its renewable properties have made PLA a popular alternative to poly(ethyleneterephthalate) (PET) for commercial applications. But no known investigation has been conducted on the use of PLA as a sustainable additive for cosmetic formulations.

Based on the foregoing, there is a need for cosmetic formulations which successfully incorporates PLA to provide deposition, hair fixing, or other benefits to the formulation.

### SUMMARY OF THE INVENTION

The invention relates to a use of a composition comprising at least one naturally derived poly(lactic acid) film-forming polymer and a carrier for styling mammalian hair.

### DETAILED DESCRIPTION OF THE INVENTION

### Cosmetic Composition

The cosmetic compositions comprise naturally derived polyesters.

It is believed that naturally derived polyesters, such as poly(lactic acid) or poly(lactide) (PLA) can provide improved cosmetic benefits such as hair fixing for hair styling compositions.

The polyesters may be included in the composition at a level by weight of from about 0.025% to about 60%, more preferably from about 0.5% to about 50%, and still more preferably from about 1% to about 15%.

In one embodiment, the compositions herein are anhydrous. Various anhydrous cosmetic compositions such as anhydrous hair care compositions and anhydrous skin care compositions can be used in the present invention. The anhydrous hair care compositions useful herein include, for example, anhydrous hair shampoo compositions, anhydrous hair styling compositions, anhydrous hair conditioning compositions, anhydrous hair color compositions, anhydrous hair growth compositions, and mixtures thereof. The anhydrous skin care compositions useful herein include, for example, anhydrous body shampoo compositions, anhydrous face cleansing compositions, anhydrous skin conditioning compositions, anhydrous shaving compositions, and mixtures thereof. As used herein, “anhydrous” means that the compositions contain 10% or less of water. The anhydrous compositions of the present invention contain, preferably 5% or less of water, more preferably 1% or less of water. Still more preferably, no water is purposely added to the anhydrous composition of the present invention.

The cosmetic compositions can be in the form of rinse-off products or leave-on products, can be transparent or opaque, and can be formulated in a wide variety of product forms, including but not limited to lotions, creams, gels, emulsions, mousses, and sprays.

A preferred form of the composition is one which forms a substantially clear (% Transmittance ≧80 at 600 nm) solution. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm is known to be adequate for characterizing the degree of clarity of cosmetic compositions.

The cosmetic compositions can be mixed with water and applied to the hair and/or skin by any conventional method well known in the art. For example, the compositions can be applied to hair and/or skin after mixing with water on hands and/or in a certain vessel. The compositions can be applied to wet hair and/or wet skin to mix with water remaining on the hair and/or skin. The compositions can also be applied to wet and/or dry hair and/or skin to mix with water when rinsed-off.

### Naturally-Derived Polyester Biopolymers

The compositions herein include naturally derived polyesters. For example, PLA is a biodegradable, thermoplastic, aliphatic polyester derived from renewable resources, such as corn starch or sugarcanes. These polymers are generally alcohol soluble, with improving solubility with decreasing molecular weight. PLA discussed herein is prepared according to the method described in WO2007/052009 to Rayner et al. Two types of PLA are particularly preferred for the compositions herein:

1) PLLA—poly(L-lactic acid), prepared from L-Lactide according to the following structure:

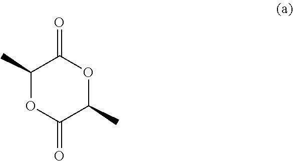

and;

2) PDLLA—poly(-DL-lactic acid), prepared from rac-lactide according to the following structure:

### Racemic Mixture of

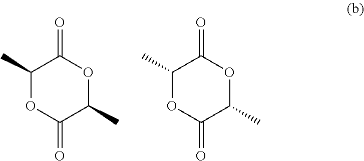

The PLLA and PDLLA polymers herein are generally alcohol soluble. Particularly preferred solvents include acetone, ethyl acetate, and ethyl lactate. In general, PLLA and PDLLA are alcohol soluble up to about 2% weight/volume in ambient conditions. In one embodiment, the compositions comprise PDLLA prepared using p-nitrobenzyl alcohol and benzyl alcohol initiators. Preparation of PLLA and PDLLA with alcohol initiators is described in detail in WO2007/052009 to Rayner et al.

### Surfactant

The compositions may also include a surfactant in an amount ranging from 0.01% to 5%, preferably from 0.01% to 1%, most preferably from 0.02% to 0.8% by weight based on total weight.

Surfactants are generally classified as nonionic, anionic, cationic, amphoteric or zwitterionic according to their ionic behavior in aqueous solution.

Examples of nonionic surfactants are condensation products of aliphatic (C₈-C₁₈) primary or secondary linear or branched chain alcohols or phenols with alkylene oxides, usually ethylene oxide and generally having from 6 to 30 ethylene oxide groups. Other suitable nonionics include esters of sorbitol, esters of sorbitan anhydrides, esters of propylene glycol, fatty acid esters of polyethylene glycol, fatty acid esters of polypropylene glycol, ethoxylated esters and polyoxyethylene fatty ether phosphates.

Examples of anionic surfactants are the alkyl sulphates, alkyl ether sulphates, alkaryl sulphonates, alkanoyl isethionates, alkyl succinates, alkyl sulphosuccinates, N-alkoyl sarcosinates, alkyl phosphates, alkyl ether phosphates, alkyl ether carboxylates, and alpha-olefin sulphonates, especially their sodium, magnesium ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from one to 10 ethylene oxide or propylene oxide units per molecule, and preferably contain 2 to 3 ethylene oxide units per molecule.

Examples of cationic surfactants are cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, (and the corresponding hydroxides thereof), and those materials having the CTFA designations Quaternium-5, Quaternium-31 and Quaternium-18.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates alkyl amidopropyl hydroxysultaines, acyl taurates and acyl glutamates, wherein the alkyl and acyl groups have from 8 to 19 carbon atoms.

The surfactants in compositions of the invention are most preferably selected from one or more nonionic surfactants. Surfactants selected from anionic, cationic, amphoteric and zwitterionic surfactants may suitably be used in conjunction with these nonionic surfactants, to improve, for example, foaming power and/or foam stability.

Particularly preferred surfactants in compositions of the invention are those nonionic surfactants selected from polyoxyethylene nonyl phenyl ether, Polysorbate 20, Polysorbate 80 and mixtures thereof, optionally in combination with one or more amphoteric surfactants. Such amphoteric surfactants are preferably selected from lauryl amine oxide, cocodimethyl sulphopropyl betaine, lauryl betaine, sodium cocamphopropionate, and especially cocamidopropyl betaine.

### Liquid Carrier

The compositions comprise a suitable liquid carrier, preferably a liquid carrier at concentrations ranging from about 40% to about 99.8%, preferably from about 50% to about 95%, more preferably from about 55% to about 80%, by weight of the composition. The liquid carrier for use herein can comprise any known or otherwise effective liquid carrier for use in cosmetic formulations intended for topical application to human hair or skin. The liquid carrier can include solvents and other optional ingredients of the hairspray compositions of the present invention.

Suitable liquid carriers for use in the anhydrous aerosol hairspray composition of the present invention include organic solvents such as C₁-C₆alkanols, carbitol, acetone, and mixtures thereof. Preferred liquid carriers are the C₁-C₆alkanols. Nonlimiting examples of preferred C₁-C₆alkanols include C₂-C₄monohydric alcohols such as ethanol, isopropanol, and mixtures thereof.

### Propellant

The compositions may comprise a nonhydrocarbon propellant suitable for aerosol delivery of the hairspray composition to the desired application surface. To provide the improved spray performance benefits, the composition should be substantially free of hydrocarbon propellants, e.g., contain less than about 5% by weight of such hydrocarbon propellants.

The compositions contain less than about 5%, preferably less than about 3%, most preferably zero percent of hydrocarbon propellants by weight of the composition. The spray performance of anhydrous aerosol hairspray compositions is improved by minimizing the concentration of the hydrocarbon propellants to less than about 5% by weight of the composition. In this context, the term “hydrocarbon propellants” as used herein refers to those liquifiable gases that contain only carbon and hydrocarbon atoms, most notably of which are propane, butane, and isobutane.

The total concentration of the nonhydrocarbon propellant in the composition can include one or more nonhydrocarbon propellants, the total nonhydrocarbon propellant concentration typically ranging from about 5% to about 50%, preferably from about 15% to about 40%, more preferably from about 25% to about 40%, by weight of the composition when used as an aerosol hairspray. In this context, the term “nonhydrocarbon propellant” refers to all liquifiable gases suitable for use in topical application to human hair or skin, excluding the above-identified hydrocarbon propellants. Nonlimiting examples of suitable nonhydrocarbon propellants include nitrogen, carbon dioxide, nitrous oxide, atmospheric gas, 1,2-difluoroethane (Hydrofluorocarbon 152A) supplied as Dymel 152A by Dupont, dimethylether, and mixtures thereof. Preferred is dimethylether.

### Water

The compositions are preferably anhydrous and therefore typically contain less than about 15%, preferably less than about 10%, more preferably less than about 5%, most preferably zero percent, of free or added water by weight of the composition.

### Optional Components

The compositions may further comprise optional components known or otherwise effective for use in hair care or personal care products, provided that the optional components are physically and chemically compatible with the essential component described herein, or do not otherwise unduly impair product stability, aesthetics or performance. The concentration of such optional ingredients generally ranges from zero to about 25%, more typically from about 0.05% to about 25%, even more typically from about 0.1% to about 15%, by weight of the composition.

Nonlimiting examples of optional ingredients include preservatives, surfactants, conditioning or styling polymers other than and in addition to the polyesters described herein, thickeners and viscosity modifiers, electrolytes, fatty alcohols, pH adjusting agents, perfume oils, perfume solubilizing agents, sequestering agents, emollients, lubricants and penetrants such as various lanolin compounds, protein hydrolysates and other protein derivatives, ethylene adducts and polyoxyethylene cholesterol, sunscreens, volatile and non-volatile silicone fluids, and isoparrafins.

The compositions may also be formulated to comprise a plasticizer at a concentration ranging from about 0.01% to about 25% by weight of the composition. The optional plasticizer is described in detail hereinafter.

### Optional Plasticizer

The compositions may further comprise an optional non-volatile plasticizer at concentrations effective to provide for improved hair style performance. Such concentrations generally range from about 0.01% to about 25%, preferably from about 0.1% to about 15%, more preferably from about 0.1% to about 5%, by weight of the composition. As used herein, the term “nonvolatile” in regard to plasticizers means that the plasticizer does not have a measurable vapor pressure under ambient conditions. The polymer-liquid carrier solution should not suffer from substantial plasticizer weight loss while the liquid carrier is evaporating, since this may excessively reduce plasticization of the polymer during use.

The optional plasticizers for use herein should generally have boiling points of from about 250° C. or higher. These materials are well known in the art and are described in Kirk-Othmer Encyclopedia of Chemical Technology, second edition, Volume 15, pp. 720-789 (John Wiley & Sons, Inc. New York, 1968) under the topic heading “Plasticizers”; in The Technology of Plasticizers, by J. Kern Sears and J. R. Darby (John Wiley & Sons, Inc., New York, 1982); and in the Appendix of Sears/Darby, Table A.9, pages 983-1063; which descriptions are incorporated herein by reference.

The optional plasticizers suitable for use in the compositions of the present invention include both cyclic and acyclic nonvolatile materials. Nonlimiting examples of suitable nonvolatile plasticizers include adipates, phthalates, isophthalates, azelates, stearates, citrates, trimellitates, silicone copolyols, iso C₁₄-C₂₂ alcohols, carbonates, sebacates, isobutyrates, oleates, phosphates, myristates, ricinoleates, pelargonates, valerates, camphor, glycols, and castor oil.

Preferred optional plasticizers for use herein include glycerin, diisobutyladipate (DIBA), glycols, and citrates. Preferred glycols include propylene glycol, dipropylene glycol and mixtures thereof. Preferred citrates include acetyl tri-n-butyl citrate, tri-n-butyl and acetyl tri-2-ethoxyhexyl citrate sold under the tradename Citroflex R™ supplied by Pfizer, and mixtures thereof.

### Method of Manufacture

Methods for preparing the compositions include conventional formulation and mixing techniques. For example, a suitable method for forming a hair spray formulation includes adding the PLA polymer to acetone and mixing for several hours until the polymer is dissolved. Then, the optional plasticizer may be then added, and the resulting solution is stirred. Any remaining ingredients such as perfume can then be added. The composition is then packaged into a suitable container such as an aerosol dispenser.

The composition can be contained or dispensed in any known or otherwise effective container or delivery system. All such containers or delivery systems should be compatible with the essential and any selected optional ingredients of the composition.

In one embodiment, pressurized aerosol dispensers can be used where the propellant and/or water and other secondary ingredients are separated from contact with the hairspray composition by use of specialized containers such as a two compartment can of the type sold under the tradename SEPRO from American National Can Corp.

Other suitable aerosol dispensers include those containing compressed air propellant which can be filled into the dispenser by means of a pump or equivalent device prior to use. Such dispensers are described in U.S. Pat. No. 4,077,441, to Olofsson, issued Mar. 7, 1978; and U.S. Pat. No. 4,850,577, to TerStege, issued Jul. 25, 1989.

### Method Of Use

The compositions may be used for any cosmetic application. In particular, the compositions are used in a conventional manner for providing hairstyle/hold benefits. An effective amount of the composition is sprayed or applied onto dry or damp hair before and/or after the hair is styled. As used herein “effective amount” means an amount sufficient to provide the hair volume and style performance desired according to the length and texture of the hair.

### Product Form

Compositions of the invention may suitably be in a pump spray or aerosol form. A particularly preferred product form is an aerosol hair mousse or spray. Aerosol hair mousse compositions are emitted from the aerosol container as a foam which is then typically worked through the hair with fingers or a hair styling tool and either left on the hair or rinsed out.

Aerosol-form compositions generally include an aerosol propellant which serves to expel the other materials from the container, and forms the mousse character in mousse compositions. The aerosol propellant can be any liquefiable gas conventionally used for aerosol containers. Examples of suitable propellants include dimethyl ether and hydrocarbon propellants such as propane, n-butane and isobutane. The propellants may be used singly or admixed. Water insoluble propellants, especially hydrocarbons, are preferred because they form emulsion droplets on agitation and create suitable mousse foam densities.

The amount of the propellant used is governed by normal factors well known in the aerosol art. For mousses the level of propellant is generally up to 30%, preferably from 2% to 5Q 30%, most preferably from 3% to 15% by weight based on total weight of the composition. If a propellant such as dimethyl ether includes a vapour pressure suppressant (e.g. trichloroethane or dichloromethane), for weight percentage calculations, the amount of suppressant is included as part of the propellant.

The method of preparing aerosol hair styling mousse compositions follows conventional aerosol filling procedures. The composition ingredients (not including the propellant) are charged into a suitable pressurisable container which is sealed and then charged with the propellant according to conventional techniques.

Compositions of the invention may also take a nonfoaming product form, such as a hair styling spray, cream, or gel. Such a cream or gel may include a structurant or thickener, 65 typically at a level of from 0.1% to 10%, preferably 0.5% to 3% by weight based on total weight.

Examples of suitable structurants or thickeners are polymeric thickeners such as carboxyvinyl polymers. A carboxyvinyl polymer is an interpolymer of a monomeric mixture comprising a monomeric olefinically unsaturated carboxylic acid, and from about 0.01% to about 10% by weight of the total monomers of a polyether of a polyhydric alcohol. Carboxyvinyl polymers are substantially insoluble in liquid, volatile organic hydrocarbons and are dimensionally stable on exposure to air. Suitably the molecular weight of the carboxyvinyl polymer is at least 750,000, preferably at least 1,250,000, most preferably at least 3,000,000. Preferred carboxyvinyl polymers are copolymers of acrylic acid crosslinked with polyallylsucrose as described in U.S. Pat. No. 2,798,053. These polymers are provided by B.F. Goodrich Company as, for example, CARBOPOL 934, 940, 941 and 980.

Natural structurants are particularly preferred, such as water soluble or colloidally water soluble polymers like cellulose ethers (e.g. methylcellulose, hydroxyethylcellulose, hydroxypropylmethylcellulose and carboxymethylcellulose), guar gum, sodium alginate, gum arabic, xanthan gum, polyvinyl alcohol, polyvinyl pyrrolidone, hydroxypropyl guar gum, starch and starch derivatives, and other thickeners, viscosity modifiers, gelling agents, etc. It is also possible to use inorganic thickeners such as bentonite or laponite clays.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Use of a composition comprising at least one naturally derived poly(lactic acid) film-forming polymer and a carrier for styling mammalian hair.

2. A use to claim 1, wherein said composition is anhydrous.

3. A use according to claim 1 or 2, wherein said composition is further characterized by one or more surfactants.

4. A use according to any one of the preceding claims, wherein said polymer is characterized by a molecular weight of from about 5 to about 50,000.

5. A use according to any one of the preceding claims, wherein said polymer is present at a level of from about 0.025% to about 60%, more preferably from about 0.5% to about 50%, and more preferably from about 1% to about 15% by weight of the composition.

6. A use according to any one of the preceding claims, wherein said composition is substantially free of synthetic polymers.

7. A use according to any one of the preceding claims, wherein said composition is substantially free of synthetic ingredients.

8. A use according to any one of the preceding claims, wherein said composition is substantially free of silicone elastomers.

9. A use according to any one of the preceding claims, said composition further comprising one or more ingredients selected from the group consisting of preservatives, conditioning or styling polymers, thickeners, electrolytes, fatty alcohols, pH adjusting agents, perfume oils, perfume solubilizing agents, sequestering agents, emollients, lubricants, penetrants, protein derivatives, ethylene adducts, sunscreens, volatile and non-volatile silicone fluids, and isoparrafins.
